(19) United States
(12) Patent Application Publication
(10) Pub. No.: US 2026/0166190 A1
(43) Pub. Date: Jun. 18, 2026

(54) SPACE DECONTAMINATION METHOD AND SPACE DECONTAMINATION DEVICE

(71) Applicant: Nippon Air Conditioning Services Co., Ltd., Nagoya-shi (JP)

(72) Inventors: Hitoshi Nakatsuka, Nagoya-shi (JP); Yuichi Nakashima, Nagoya-shi (JP)

(21) Appl. No.: 18/842,942

(22) PCT Filed: Nov. 17, 2023
(86) PCT No.: PCT/JP2023/041455

§ 371 Date: Aug. 30, 2024

(30) Foreign Application Priority Data
Jun. 30, 2023 (JP) 2023-108419

(51) Int. Cl. A61L 2/208; A61L 9/12; A61L 101/02; A61L 101/36

(52) U.S. Cl. CPC A61L 2/208; A61L 9/12; A61L 2101/02; A61L 2101/36

(57) ABSTRACT

According to a space decontamination method, a mixed gas containing air and acetic acid is supplied to a space, and humidify the space by using an aqueous solution containing hydrogen peroxide. The space decontamination device includes a mixed gas supply unit and a humidifying unit. The mixed gas supply unit supplies the mixed gas containing air and acetic acid to the space. The humidifying unit humidifies the space by using an aqueous solution containing hydrogen peroxide. The mixed gas supply unit generates the mixed gas by sending air into a solution containing acetic acid and performing bubbling, for example.

### CROSS-REFERENCE TO RELATED APPLICATION

This international application claims the benefit of Japanese Patent Application No. 2023-108419 filed on Jun. 30, 2023 with the Japan Patent Office, and the entire disclosure of Japanese Patent Application No. 2023-108419 is incorporated herein by reference.

### TECHNICAL FIELD

The present disclosure relates to a space decontamination method and a space decontamination device.

### BACKGROUND ART

Facilities such as drug manufacturing facilities, biohazardous facilities, and cell culturing and processing facilities have spaces that need to be decontaminated. A method of decontamination is disclosed in Patent Document 1. One method of decontamination is to spray a solution including peracetic acid into a space using a spraying device.

### PRIOR ART DOCUMENTS

### Patent Documents

Patent Document 1: Published Japanese Translation of PCT International Publication for Patent Application No. 2005-526557

### SUMMARY OF THE INVENTION

### Problems to be Solved by the Invention

A space decontamination method that achieves more effective decontamination is desired. In one aspect of the present disclosure, it is preferable to provide a space decontamination method and a space decontamination device that achieve highly effective decontamination.

### Means for Solving the Problems

One aspect of the present disclosure is a space decontamination method that includes supplying mixed gas containing air and acetic acid to a space, and humidifying the space with an aqueous solution containing hydrogen peroxide. The space decontamination method in one aspect of the present disclosure achieves highly effective decontamination.

Another aspect of the present disclosure is a space decontamination device that includes a mixed gas supply unit configured to supply mixed gas containing air and acetic acid to a space, and a humidifying unit configured to humidify the space with an aqueous solution containing hydrogen peroxide. The space decontamination device in another aspect of the present disclosure achieves highly effective decontamination.

### BRIEF DESCRIPTION OF THE DRAWINGS

FIG. 1 is an explanatory diagram showing a configuration of a mixed gas supply unit.

FIG. 2 is a block diagram showing a configuration of a space decontamination device.

FIG. 3 is an explanatory diagram showing a small-sized booth and the space decontamination device.

FIG. 4 is a graph showing transitions of concentration of acetic acid, concentration of hydrogen peroxide, temperature, and humidity of Example 1.

FIG. 5 is a graph showing transitions of concentration of acetic acid, temperature, and humidity of Comparative Example 1.

FIG. 6 is an explanatory diagram showing a safety cabinet and the space decontamination device.

FIG. 7 is an explanatory diagram showing circulation paths of air and locations to place a BI in the safety cabinet.

FIG. 8 is a graph showing transitions of concentration of acetic acid, concentration of hydrogen peroxide, temperature, and humidity of Example 2.

FIG. 9 is a graph showing transitions of concentration of hydrogen peroxide, temperature, and humidity of Comparative Example 2.

FIG. 10 is an explanatory diagram showing a clean room and the space decontamination device.

FIG. 11 is a graph showing transitions of concentration of acetic acid, concentration of hydrogen peroxide, temperature, and humidity of Example 3.

FIG. 12 is a graph showing transitions of concentration of hydrogen peroxide, temperature, and humidity of Comparative Example 3.

FIG. 13 is an explanatory diagram showing a microorganism examination room and the space decontamination device.

FIG. 14 is a graph showing transitions of concentration of acetic acid, concentration of hydrogen peroxide, temperature, and humidity of Example 4.

### EXPLANATION OF REFERENCE NUMERALS

1 . . . mixed gas supply unit; 13 . . . container; 15 . . . trap; 17, 19, 21 . . . pipe; 17A . . . leading end; 18 . . . air; 20 . . . safety cabinet; 23 . . . first solution; 25 . . . mixed gas; 101 . . . space decontamination device; 103 . . . humidifying unit; 105 . . . space; 107 . . . control unit; 107A . . . CPU, 107B . . . memory; 109 . . . information acquisition unit; 111 . . . small-sized booth; 201 . . . safety cabinet; 203 . . . workbench; 205 . . . work space; 207 . . . front opening; 209 . . . fan room; 211 . . . ceiling air outlet; 215 . . . circulator; 217, 219 . . . duct; 221 . . . back side pathway; 223 . . . air pump; 225 . . . circulation fan; 301 . . . clean room; 303 . . . front chamber; 305 . . . door; 307 . . . dehumidifier; 309 . . . dehumidifier terminal box; 401 . . . microorganism examination room; 403, 405 . . . laboratory table; 407 . . . sink.

### MODE FOR CARRYING OUT THE INVENTION

Exemplary embodiments of the present disclosure will be described hereinafter with reference to the accompanying drawings.

### 1. SPACE DECONTAMINATION METHOD

A space decontamination method according to the present disclosure is for decontaminating a space. Examples of the space are those located in drug manufacturing facilities, biohazardous facilities, and cell culturing and processing facilities. Examples of the space are those located inside a safety cabinet, an isolator, and a Restricted Access Barrier System. Examples of the space are those in a clean area and those in an aseptic operation area. The spaces in the clean area and the aseptic operation area are located in food factories, for example. The space includes, for example, walls, floors, ceilings, and equipment located at the boundaries between the space and other areas.

Decontamination means reducing microorganisms that exist in the space. Decontamination means reducing microorganisms that exist in the space to a predesignated bacteria count level, for example. Decontamination is reducing BI by 6 log or more, for example. The BI stands for biological indicator, which means an indicator bacterium.

In the space decontamination method of the present disclosure, mixed gas containing air and acetic acid is supplied into the space. In the space decontamination method of the present disclosure, mixed gas is generated by sending air into a solution containing acetic acid (hereinafter referred to as a first solution) and performing bubbling, for example. The first solution is an aqueous solution of acetic acid, for example. The concentration of acetic acid in the first solution is preferably 5 w/v % or higher and 50 w/v % or lower. In a case where the concentration of acetic acid in the first solution is 5 w/v % or higher, the effect of decontaminating the space is even higher. In a case where the concentration of acetic acid in the first solution is 50 w/v % or lower, safety when performing the space decontamination method is even higher. For example, solutions containing peracetic acid and hydrogen peroxide are excluded from the first solution even if they contain acetic acid. In other words, a solution containing acetic acid, peracetic acid, and hydrogen peroxide is not the first solution, for example.

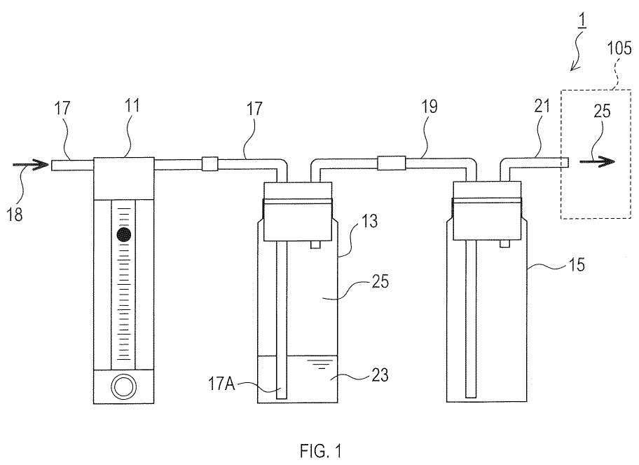

The mixed gas may further contain water vapor in addition to air and acetic acid. One of the methods to generate the mixed gas and supply the mixed gas into the space is to use a mixed gas supply unit 1 shown in FIG. 1.

The mixed gas supply unit 1 includes a flowmeter 11, a container 13, a trap 15, and pipes 17, 19, 21. The pipe 17 sends air 18 supplied from an air supply source, which is not illustrated, inside the container 13 via the flowmeter 11. The flowmeter 11 measures the flow rate of the air 18 delivered inside the container 13 and displays the flow rate. The flow rate of the air 18 is 1 L/min or greater and 5 L/min or less, for example.

A first solution 23 is stored inside the container 13. A leading end 17A of the pipe 17 is in the first solution 23. Thus, the air 18 discharged from the leading end 17A causes bubbling. Mixed gas 25 is generated in a portion of the container 13 above the surface of the first solution 23. The mixed gas 25 includes bubbles of air that passed through the first solution 23. The mixed gas 25 contains air and acetic acid. The mixed gas 25 also contains a small amount of water vapor.

The mixed gas 25 flows from the container 13 to the trap 15 via the pipe 19. The trap 15 is an empty container. A water droplet precipitated from the mixed gas 25 is removed in the trap 15. The mixed gas 25 flows from the trap 15, through the pipe 21, to a space 105 in which the space decontamination method is performed.

In the space decontamination method according to the present disclosure, the space 105 is humidified by using an aqueous solution containing hydrogen peroxide (hereinafter referred to as second solution). The concentration of hydrogen peroxide in the second solution is preferably 2 w/v % or higher and 6 w/v % or lower. In a case where the concentration of hydrogen peroxide is 2 w/v % or higher, the effect of decontaminating the space 105 is even higher. In a case where the concentration of hydrogen peroxide is 6 w/v % or lower, safety when performing the space decontamination method is even higher.

For example, the space 105 may be humidified by vaporizing the second solution by using a humidifier. A commercially available product can be used as the humidifier. Types of the humidifier may include ultrasonic type, steam type, vaporizing type, and hybrid type.

The space 105 may be humidified by spraying the second solution by using a sprayer, a spray nozzle, or a humidifier, for example. Examples of the sprayer may include an electric sprayer used for spraying disinfectant. Examples of the humidifier may include an ultrasonic type humidifier.

It is preferable that the humidity of the space 105 is 60% RH or higher and 80% RH or lower when performing the space decontamination method according to the present disclosure. In a case where the humidity of the space 105 is 60% RH or higher, the effect of decontaminating the space 105 is even higher. In a case where the humidity of the space 105 is 80% RH or lower, condensation can be further reduced.

Humidity of the space 105 can be measured by using a humidity sensor, for example, when performing the space decontamination method according to the present disclosure. For example, the humidity of the space 105 can be adjusted based on the measured value of humidity. For example, the humidity of the space 105 can be adjusted by controlling the humidifier and the nozzle based on the measured value of humidity.

The concentration of acetic acid in the space 105 is preferably 10 ppm or higher and 100 ppm or lower, and more preferably 10 ppm or higher and 70 ppm or lower when performing the space decontamination method according to the present disclosure. In a case where the concentration of acetic acid in the space 105 is 10 ppm or higher, the effect of decontaminating the space 105 is even higher. In a case where the concentration of acetic acid in the space 105 is 100 ppm or lower, gas treatment after the decontamination is simplified. In a case where the concentration of acetic acid in the space 105 is 70 ppm or lower, the gas treatment after the decontamination is further simplified. The concentration of acetic acid in the space 105 increases as the amount of supply of the mixed gas 25 to the space 105 increases. The concentration of acetic acid in the space 105 increases as the concentration of acetic acid in the first solution 23 increases. The method of measuring the concentration of acetic acid in the space 105 is a method using a detector tube.

The concentration of hydrogen peroxide in the space 105 is preferably 10 ppm or higher and 60 ppm or lower when performing the space decontamination method according to the present disclosure. In a case where the concentration of hydrogen peroxide in the space 105 is 10 ppm or higher, the effect of decontaminating the space 105 is even higher. In a case where the concentration of hydrogen peroxide in the space 105 is 60 ppm or lower, safety when performing the space decontamination method is even higher. The concentration of hydrogen peroxide in the space 105 increases as the concentration of hydrogen peroxide in the second solution increases. The concentration of hydrogen peroxide in the space 105 and humidity of the space 105 increase as humidification using the second solution is facilitated.

The method of measuring the concentration of hydrogen peroxide in the space 105 is a controlled potential electrolysis method. A controlled potential electrolysis portable gas detector is used to measure the concentration of hydrogen peroxide. In the space decontamination method according to the present disclosure, for example, the space 105 is humidified by using the second solution at the same time as supplying the mixed gas 25 into the space 105. In this case, the effect of decontaminating the space 105 is even higher.

The concentration of peracetic acid in the space 105 is preferably 1 ppm or lower when performing the space decontamination method according to the present disclosure. In this case, safety when performing the space decontamination method is even higher.

### 2. SPACE DECONTAMINATION DEVICE 101

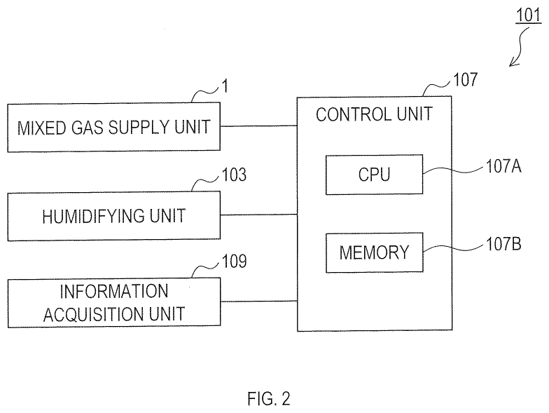

A space decontamination device 101 according to the present disclosure includes a configuration as shown in FIG. 2. The space decontamination device 101 includes a mixed gas supply unit 1. The mixed gas supply unit 1 generates the mixed gas 25 containing air and acetic acid and supply the mixed gas 25 into the space 105.

Examples of the mixed gas supply unit 1 may include a unit shown in FIG. 1. The configuration of the mixed gas supply unit 1 shown in FIG. 1 is the same as the configuration explained in the aforementioned “1. Space Decontamination Method”.

The space decontamination device 101 according to the present disclosure is used to perform the space decontamination method according to the present disclosure, for example. The first solution 23 and the mixed gas 25 are the same as those explained in the aforementioned “1. Space Decontamination Method”, for example.

The space decontamination device 101 includes a humidifying unit 103. The humidifying unit 103 humidifies the space 105 by using the second solution. The humidifying unit 103 humidifies the space 105 by vaporizing the second solution by using the humidifier, for example. A commercially available product can be used as the humidifier. Types of the humidifier may include ultrasonic type, steam type, vaporizing type, and hybrid type. The second solution is those described in the aforementioned “1. Space Decontamination Method”, for example.

The humidifying unit 103 humidifies the space 105 by spraying the second solution by using a sprayer, a spray nozzle, or a humidifier, for example. Examples of the sprayer may include an electric sprayer used for spraying disinfectant. Examples of the humidifier may include an ultrasonic type humidifier.

The space decontamination device 101 further includes a control unit 107 configured to control at least one of the mixed gas supply unit 1 or the humidifying unit 103, for example. The control unit 107 includes a microcomputer including a CPU 107A and a semiconductor memory such as a RAM and a ROM (hereinafter referred to as memory 107B), for example.

Functions of the control unit 107 are each achieved by the CPU 107A executing a program stored in a non-transitory tangible storage medium. In this example, the memory 107B is the non-transitory tangible storage medium storing the program. Execution of this program also causes a method corresponding to the program to be performed. The control unit 107 may include a single microcomputer or two or more microcomputers.

The space decontamination device 101 further includes an information acquisition unit 109, for example. The information acquisition unit 109 is configured to acquire information (hereinafter referred to as spatial information) representing one or more items selected from a group consisting of the humidity of the space 105, the temperature of the space 105, the concentration of acetic acid in the space 105, and the concentration of hydrogen peroxide in the space 105. The information acquisition unit 109 includes a sensor that can detect the spatial information, for example.

The control unit 107 controls at least one of the mixed gas supply unit 1 or the humidifying unit 103 based on the spatial information obtained by the information acquisition unit 109, for example.

The control unit 107 brings the humidity of the space 105 to 60% RH or higher and 80% RH or lower by controlling the humidifying unit 103 based on the spatial information obtained by the information acquisition unit 109, for example. In a case where the humidity of the space 105 is 60% RH or higher, the effect of decontaminating the space 105 is even higher. In a case where the humidity of the space 105 is 80% RH or lower, condensation can be further reduced. In a case where the humidity of the space 105 is 75% RH or lower, condensation can be particularly reduced.

The control unit 107 brings the concentration of acetic acid in the space 105 to 10 ppm or higher and 100 ppm or lower by controlling the mixed gas supply unit 1 based on the spatial information obtained by the information acquisition unit 109, for example. In a case where the concentration of acetic acid in the space 105 is 10 ppm or higher, the effect of decontaminating the space 105 is even higher. In a case where the concentration of acetic acid in the space 105 is 100 ppm or lower, the gas treatment after the decontamination is simplified. The control unit 107 brings the concentration of acetic acid in the space 105 to 10 ppm or higher and 70 ppm or lower, for example. In a case where the concentration of acetic acid in the space 105 is 70 ppm or lower, the gas treatment after the decontamination is further simplified.

The control unit 107 brings the concentration of hydrogen peroxide in the space 105 to 10 ppm or higher and 100 ppm or lower by controlling the humidifying unit 103 based on the spatial information obtained by the information acquisition unit 109, for example. In a case where the concentration of hydrogen peroxide in the space 105 is 10 ppm or higher, the effect of decontaminating the space 105 is even higher. In a case where the concentration of hydrogen peroxide in the space 105 is 100 ppm or lower, safety when performing the space decontamination method is even higher. The control unit 107 brings the concentration of hydrogen peroxide in the space 105 to 10 ppm or higher and 70 ppm or lower, for example. In a case where the concentration of hydrogen peroxide in the space 105 is 70 ppm or lower, safety when performing the space decontamination method is particularly high.

The space decontamination device 101 according to the present disclosure humidifies the space 105 by using the second solution at the same time as supplying the mixed gas into the space 105, for example. In this case, the effect of decontaminating the space 105 is even higher.

The space decontamination device 101 according to the present disclosure further includes a dehumidifying unit, for example. The dehumidifying unit lowers the humidity of the space 105. For example, by lowering the humidity of the space 105 by using the dehumidifying unit while humidifying the space 105 by using the humidifying unit 103, the concentration of hydrogen peroxide in the space 105 can be increased while reducing the humidity in the space 105. For example, the control unit 107 can control the dehumidifying unit based on the spatial information so that the humidity and the concentration of hydrogen peroxide in the space 105 are within a preferable range.

For example, the space decontamination method can be performed after dehumidifying the space 105 by using the dehumidifying unit. Alternatively, for example, the space decontamination method can be performed while dehumidifying the space 105 by using the dehumidifying unit. The dehumidifying unit may be operated intermittently or continuously.

### 3. EFFECTS OF SPACE DECONTAMINATION METHOD AND SPACE DECONTAMINATION DEVICE 101

(1A) The space decontamination method and the space decontamination device 101 according to the present disclosure achieve high decontamination effects.

(1B) By using the space decontamination method and the space decontamination device 101 according to the present disclosure, the space 105 can be effectively decontaminated while reducing the humidity of the space 105. The reasons are as follows. The mixed gas 25 contains a large amount of acetic acid while containing a small amount of water vapor. Thus, the concentration of acetic acid in the space 105 can be increased while reducing the humidity of the space 105 by supplying the mixed gas 25 into the space 105. In addition, the humidity of the space 105 can be moderately increased while increasing the concentration of hydrogen peroxide in the space 105 by humidifying the space 105 with the second solution. By reducing the humidification using the second solution, the humidity of the space 105 can be reduced without lowering the concentration of acetic acid in the space 105. Consequently, the space 105 can be effectively decontaminated due to a synergistic effect of acetic acid and hydrogen peroxide while reducing the humidity of the space 105.

(1C) The space decontamination method according to the present disclosure is even safer than decontamination methods using peracetic acid and formaldehyde. The space decontamination method according to the present disclosure can reduce the concentration of hydrogen peroxide in the space 105 while maintaining the effect of decontamination. Consequently, the gas treatment after the decontamination is simple.

(1D) Since acetic acid and hydrogen peroxide decompose in a short time, it is highly safe even in a case where gas containing acetic acid and hydrogen peroxide leaks out from the space 105.

(1E) Gas containing acetic acid and hydrogen peroxide has high permeability. Accordingly, even when there is an area where air does not circulate well in the space 105, it is still possible to decontaminate such an area.

### 4. EXAMPLES

### (4-1) Example 1

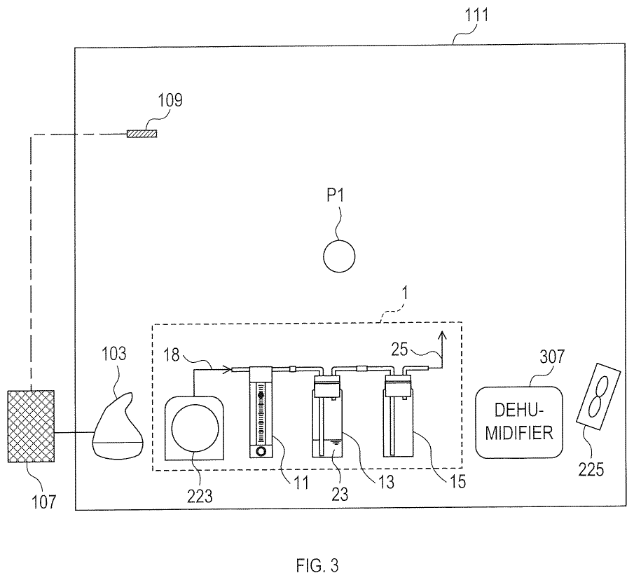

A small-sized booth 111 as shown in FIG. 3 was prepared. The volume of the small-sized booth 111 was 1 m³. The interior of the small-sized booth 111 corresponds to the space 105. The mixed gas supply unit 1, a circulation fan 225, the humidifying unit 103, the information acquisition unit 109, and a dehumidifier 307 were disposed in the interior of the small-sized booth 111.

The mixed gas supply unit 1 included an air pump 223. The air pump 223 was configured to take the air 18 in from the interior of the small-sized booth 111 and supply thus taken air 18 to the flowmeter 11. The flow rate of the air 18 was 5 L/min. The first solution 23 was an aqueous solution containing 6 w/v % of acetic acid. The amount of the first solution 23 was 40 ml.

The position where the mixed gas supply unit 1 supplies the mixed gas 25 was anterior to the circulation fan 225. The mixed gas 25 supplied by the mixed gas supply unit 1 was diffused by wind generated by the circulation fan 225.

The humidifying unit 103 was a humidifier. The interior of the small-sized booth 111 was humidified by the humidifying unit 103. The second solution was an aqueous solution containing 2 w/v % of hydrogen peroxide.

In the present Example, the information acquisition unit 109 was a temperature and humidity sensor. The information acquisition unit 109 detected the temperature and humidity of air in the interior of the small-sized booth 111 and transmitted the detection results to the control unit 107. The control unit 107 controlled the humidifying unit 103 based on the results of the measurement of the temperature and humidity. The control unit 107 controlled the humidity of the interior of the small-sized booth 111 to be about 75% RH.

BI were disposed at the location of P1 shown in FIG. 3. P1 was situated near the center of the small-sized booth 111. The BI were indicator bacteria (Geobacillus stearothermophilus (ATCC #12980)) manufactured by Mesa Labs. The product number was HMV-091. The bacteria count of the BI was 1×10⁶ or greater. The BI were prepared for hydrogen peroxide.

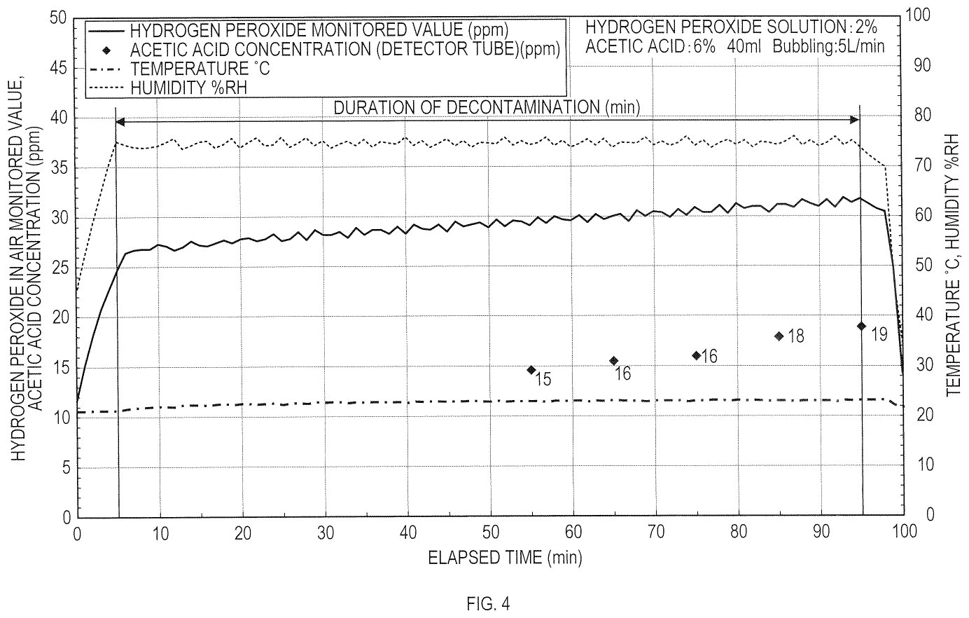

The space decontamination method was performed by supplying the mixed gas 25 into the small-sized booth 111 by using the mixed gas supply unit 1 and humidifying the small-sized booth 111 by using the humidifying unit 103. FIG. 4 shows transitions of the concentration of hydrogen peroxide, the concentration of acetic acid, temperature, and humidity in the interior of the small-sized booth 111 when performing the space decontamination method. The horizontal axis of FIG. 4 represents time elapsed since the time humidification and supply of the mixed gas 25 were initiated.

The dehumidifier 307 was continuously operated when performing the space decontamination method. When an attempt was made to control the humidity at about 75% RH when the dehumidifier 307 was operated continuously, the amount of spray by the humidifying unit 103 increased compared with a case where the dehumidifier 307 was not operated. Although the life of hydrogen peroxide in the atmosphere is short, the concentration of hydrogen peroxide in the small-sized booth 111 can be increased by increasing the amount of spray by the humidifying unit 103

The BI were respectively recovered from the location of P1 at 70 minutes, 80 minutes, and 90 minutes of decontamination time. The decontamination time is a time elapsed since the time at which the humidity of the interior of the small-sized booth 111 reached 75% RH. Then, the recovered BI were cultivated for seven days in dedicated culture media under a proper temperature to confirm whether the BI were negative or positive. If it is negative, the BI were killed. If it is positive, the BI were not killed. The results were shown in Table 1.

TABLE 1

| | Decontamination Time (min) | | |
|---|---|---|---|
| BI | 70 | 80 | 90 |
| P1 | + | + | − |

(+positive −negative)

The BI recovered at 90 minutes of the decontamination time was negative. This result confirms that decontamination of 90 minutes can achieve decontamination level of 6 log or greater. During the period of 50 to 90 minutes of the decontamination time, the average concentration of acetic acid was 17 ppm and the average concentration of hydrogen peroxide was 31 ppm in the interior of the small-sized booth 111.

When performing the space decontamination method, the concentration of peracetic acid in the interior of the small-sized booth 111 was measured. The concentration of peracetic acid was measured by retrieving a sample with an impinger, and measuring the concentration of peracetic acid in the retrieved sample by MTS oxidation GC-FID analysis method. The concentration of peracetic acid was 0.04 ppm or lower, which was the lower detection limit. Accordingly, it was able to confirm that peracetic acid did not contribute to decontamination. The concentration of peracetic acid was at or below the lower detection limit in the examples described later.

### (4-2) Comparative Example 1

Decontamination was performed basically in the same manner as in Example 1. However, in Comparative Example 1, the second solution was not an aqueous solution of hydrogen peroxide but pure water. Thus, in Comparative Example 1, hydrogen peroxide was not supplied to the interior of the small-sized booth 111. In addition, in Comparative Example 1, the first solution 23 was an aqueous solution containing 12 w/v % of acetic acid.

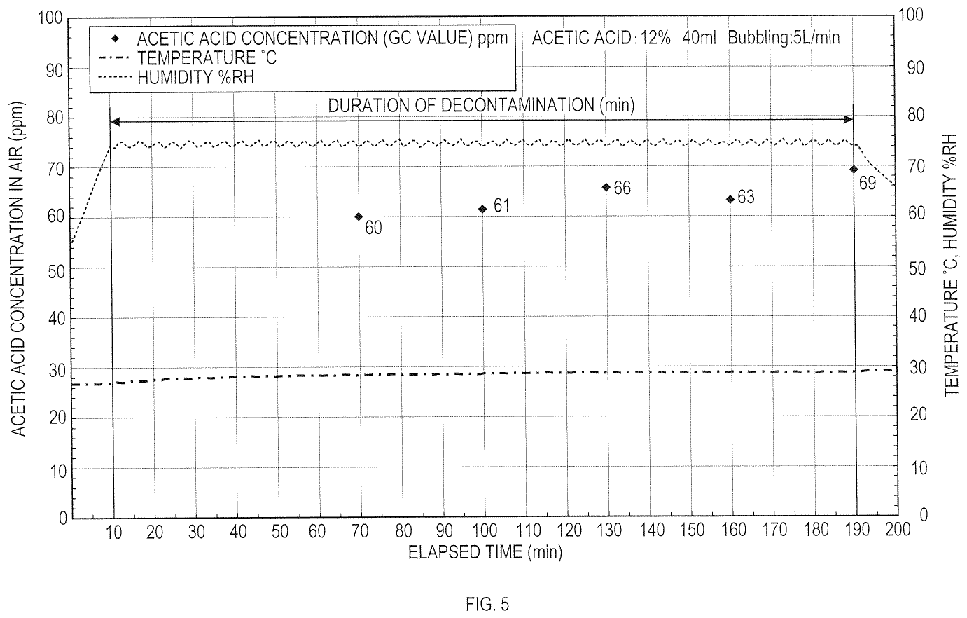

FIG. 5 shows transitions of the concentration of acetic acid, temperature, and humidity in the interior of the small-sized booth 111 when performing the space decontamination method. The horizontal axis of FIG. 5 represents time elapsed since the time humidification and supply of the mixed gas 25 were initiated. The BI were respectively recovered from the location of P1 at 90 minutes and 180 minutes of the decontamination time. The decontamination time is a time elapsed since the time at which the humidity of the interior of the small-sized booth 111 reached 75% RH. Then, the recovered BI were cultivated for seven days in the dedicated culture media under a proper temperature to confirm whether the BI were negative or positive. The results were shown in Table 2.

TABLE 2

Decontamination Time (min)

| BI | 90 | 180 |
|---|---|---|
| P1 | + | + |

(+positive −negative)

As shown in Table 2, both of the BI were positive and thus the BI were not killed. During the period of 60 to 180 minutes of the decontamination time, the average concentration of acetic acid was 64 ppm in the interior of the small-sized booth 111, which was notably higher than the average concentration of acetic acid in Example 1, however, the effect of decontamination was low. This result shows that decontamination is difficult when only acetic acid is supplied into the space 105.

### (4-3) Example 2

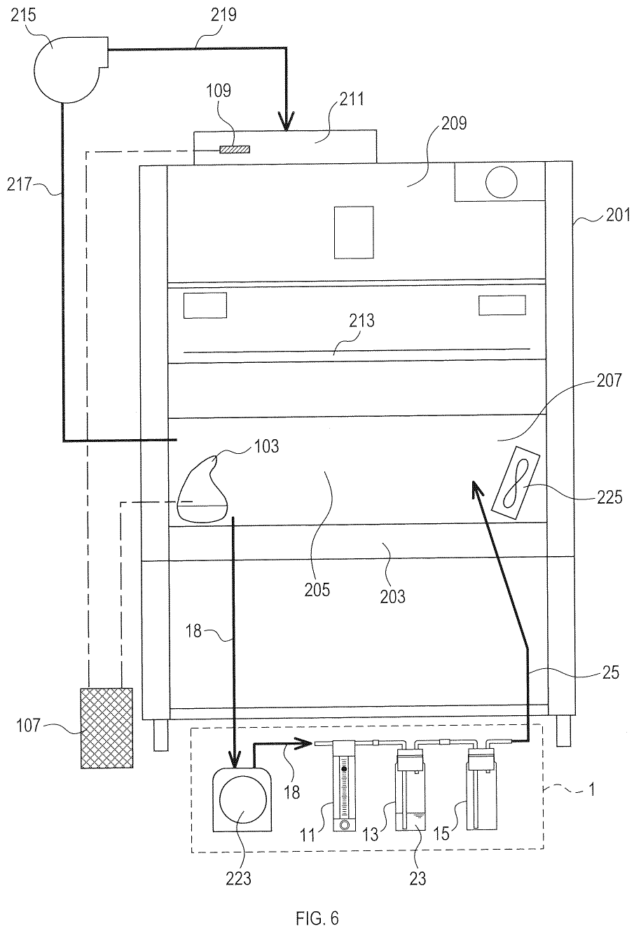

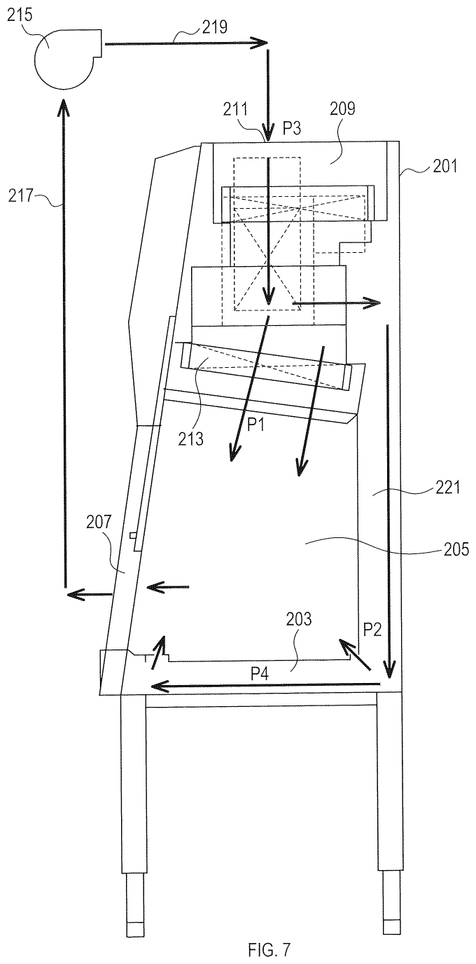

A safety cabinet 201 as shown in FIG. 6 and FIG. 7 was prepared. The safety cabinet 201 is used in biohazardous facilities handling highly infectious microorganisms, for example. The safety cabinet 201 is also used in cell processing or the like that requires sterility in the technical field of regenerative medicine, for example.

The safety cabinet 201 is a device that requires a high decontamination level of 6 log or higher, for example. The interior of the safety cabinet 201 corresponds to the space 105. The width of the opening of the safety cabinet 201 was 1.3 m. The safety cabinet 201 was a class II anti-biohazard cabinet.

The safety cabinet 201 includes a workbench 203. A work space 205 is situated above the workbench 203. The safety cabinet 201 includes a front opening 207. The work space 205 is situated at the back side of the front opening 207 as seen from the front side. The safety cabinet 201 includes a fan room 209, a ceiling air outlet 211, and a HEPA 213.

As shown in FIG. 6 and FIG. 7, a circulator 215 disposed outside the safety cabinet 201 and the front opening 207 were coupled to each other via a duct 217. The ceiling air outlet 211 and the circulator 215 were coupled to each other via a duct 219. The front opening 207 was sealed with curing tapes and a vinyl chloride panel except for the part connected with the duct 217. The ceiling air outlet 211 was also sealed with curing tapes and a vinyl chloride panel except for the part connected with the duct 219. Other gaps in the safety cabinet 201 were also sealed with curing tapes.

When the circulator 215 is activated, air circulation occurs along the path shown with arrows in FIG. 7. Air in the work space 205 flows out from the front opening 207, passes through the duct 217, the circulator 215, the duct 219, and the ceiling air outlet 211 in this order and enters the fan room 209. A part of the air in the fan room 209 passes through the HEPA 213 and enters the work space 205. A part of the air in the fan room 209 passes through a back side pathway 221 and enters the work space 205 from the back side of the workbench 203.

As shown in FIG. 6, the mixed gas 25 generated in the mixed gas supply unit 1 was supplied to the work space 205. The mixed gas supply unit 1 included the air pump 223. The air pump 223 takes the air 18 in from the work space 205 and supplies thus taken air 18 to the flowmeter 11. The flow rate of the air 18 was 5 L/min. The first solution 23 was an aqueous solution containing 40 w/v % of acetic acid. The amount of the first solution 23 was 40 ml.

As shown in FIG. 6, the circulation fan 225 was disposed on the workbench 203. The position where the mixed gas 25 was supplied was anterior to the circulation fan 225. The mixed gas 25 supplied by the mixed gas supply unit 1 was diffused by wind generated by the circulation fan 225 and sent to the duct 217.

As shown in FIG. 6, the humidifying unit 103 was placed on the workbench 203, and the work space 205 was humidified by using the humidifying unit 103. The second solution was an aqueous solution containing 3 w/v % of hydrogen peroxide. In the present Example, the information acquisition unit 109 was a temperature and humidity sensor. The information acquisition unit 109 detected the temperature and humidity of air at the ceiling air outlet 211 and transmitted the results of detection to the control unit 107. The control unit 107 controlled the humidifying unit 103 based on the results of the measurement of the temperature and humidity. The control unit 107 controlled the humidity in the safety cabinet 201 to be about 75% RH.

The BI were placed at the locations of P1 to P4 shown in FIG. 7. P1 was situated in the work space 205 near the HEPA 213. P2 was situated at the bottom of the interior of the back side pathway 221. P3 was situated at a perforated metal disposed at the ceiling air outlet 211. P4 was situated at a lower part of the workbench 203.

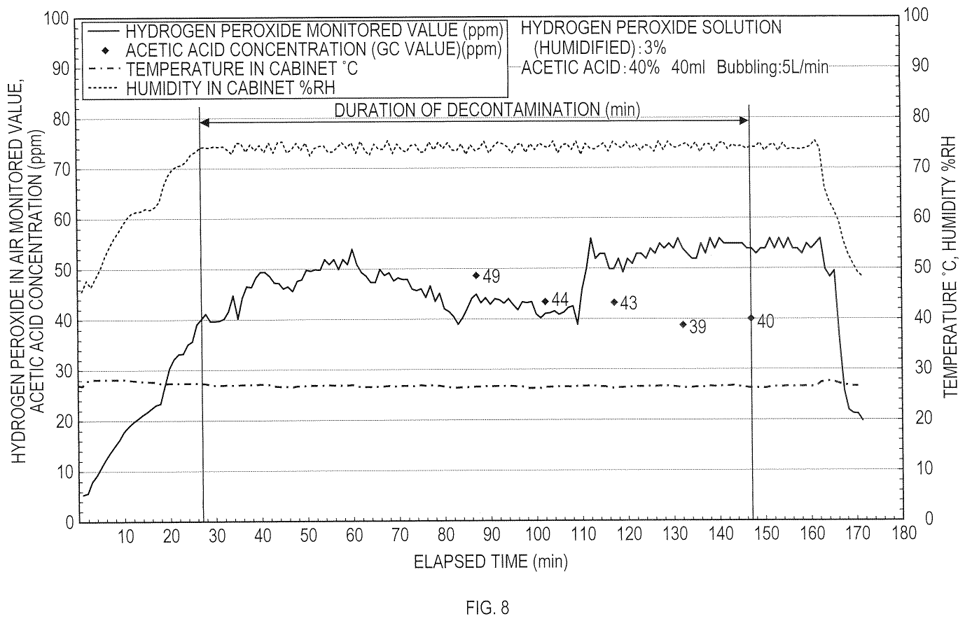

The BI were Geobacillus stearothermophilus (ATCC #12980). The bacteria count of the BI was 1×10⁶ or greater. Decontamination was initiated in a state where air is circulated in the safety cabinet 201 as shown in FIG. 7. The decontamination is to supply the mixed gas 25 into a space by using the mixed gas supply unit 1 and to humidify the space by using the humidifying unit 103. FIG. 8 shows transitions of concentration of acetic acid, concentration of hydrogen peroxide, temperature, and humidity in the safety cabinet 201 during decontamination. The horizontal axis of FIG. 8 represents time elapsed since the time humidification and supply of the mixed gas 25 were initiated.

The BI were respectively recovered from the locations of P1, P2, and P3 at 120 minutes of the decontamination time. The decontamination time is a time elapsed since the time at which the humidity of the interior of the safety cabinet 201 reached 75% RH. The BI were recovered from the location of P4 respectively at 60 minutes, 75 minutes, 90 minutes, 105 minutes, and 120 minutes of the decontamination time. Then, the recovered BI were cultivated for seven days in dedicated culture media under a proper temperature to confirm whether the BI were negative or positive. The results were shown in Table 3. Three BI were recovered for one condition. The condition here is a combination of the location of the BI and the decontamination time when recovering the BI. If two or more BI out of three BI were negative, it was determined that decontamination in this particular condition was successful.

TABLE 3

| | Decontamination Time (min) | | | | |
|---|---|---|---|---|---|
| BI | 60 | 75 | 90 | 105 | 120 |
| P1 | | | | | − |
| | | | | | − |
| | | | | | − |
| P2 | | | | | − |
| | | | | | − |
| | | | | | − |
| P3 | | | | | − |
| | | | | | − |
| | | | | | − |
| P4 | − | − | − | + | − |
| | − | − | − | − | − |
| | − | − | − | − | − |

(+positive −negative)

As shown in Table 3, decontamination was successful at all of the locations P1 to P4 and all of the decontamination times.

### (4-4) Comparative Example 2

Decontamination was performed basically in the same manner as in Example 2. However, in Comparative Example 2, the mixed gas 25 was not supplied to the work space 205. In other words, in Comparative Example 2, acetic acid was not supplied to the work space 205. The BI were placed at the location of P4.

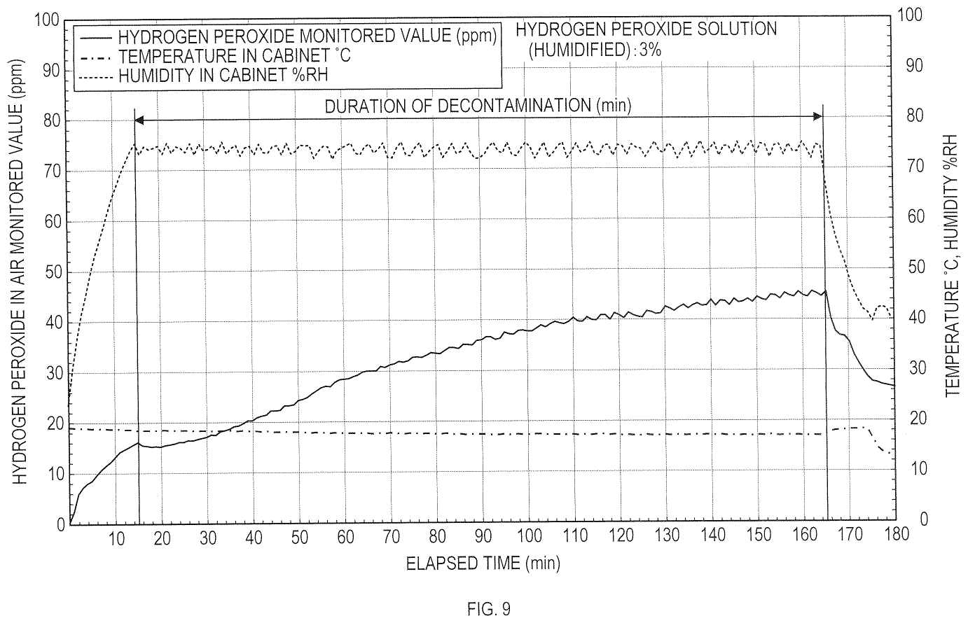

FIG. 9 shows transitions of concentration of hydrogen peroxide, temperature, and humidity in the safety cabinet 201 during the decontamination. The horizontal axis of FIG. 9 represents time elapsed since the time humidification was initiated.

The BI were respectively recovered from the locations of P1, P2, and P3 at 150 minutes of the decontamination time. The decontamination time is a time elapsed since the time at which the humidity of the interior of the safety cabinet 201 reached 75% RH. The BI were also recovered from the location of P4 respectively at 80 minutes, 90 minutes, 100 minutes, 110 minutes, 120 minutes, 130 minutes, 140 minutes, and 150 minutes of the decontamination time. Then, the recovered BI were cultivated for seven days in the dedicated culture media under a proper temperature to confirm whether the BI were negative or positive. The results were shown in Table 4. One BI was recovered for one condition.

TABLE 4

| | Decontamination Time (min) | | | | | | | |
|---|---|---|---|---|---|---|---|---|
| BI | 80 | 90 | 100 | 110 | 120 | 130 | 140 | 150 |
| P1 | | | | | | | | + |
| P2 | | | | | | | | + |
| P3 | | | | | | | | + |
| P4 | + | + | + | + | + | + | + | + |

(+positive −negative)

As shown in Table 4, the BI were positive at all of the locations of P1 to P4 and at all of the decontamination times. This results indicate that it is difficult to achieve decontamination when only hydrogen peroxide is supplied to the work space 205.

### (4-5) Example 3

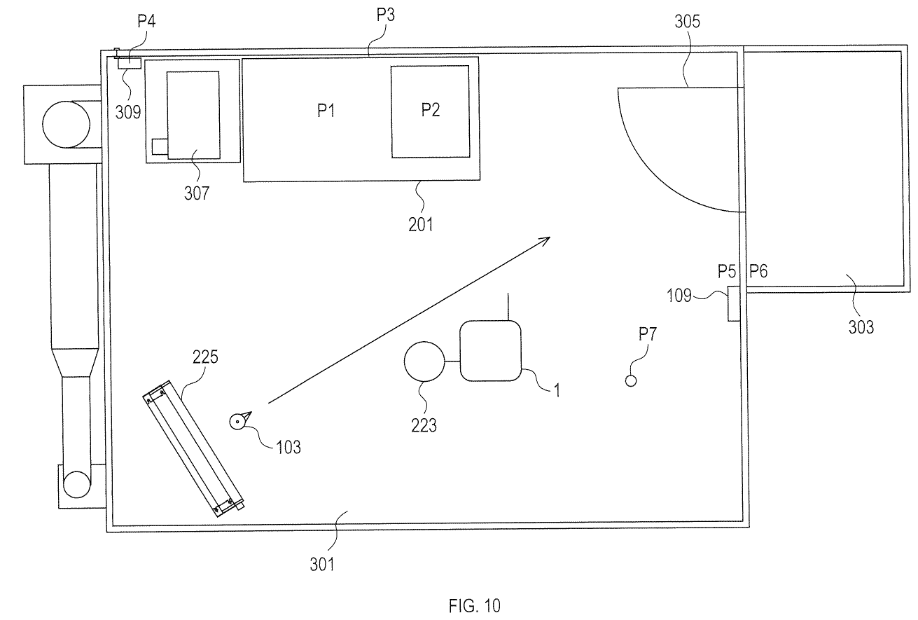

The space decontamination method was performed in a clean room 301 and a front chamber 303 shown in FIG. 10. A door 305 was situated between the clean room 301 and the front chamber 303. The door 305 was open at all times. The total volume of the clean room 301 and the front chamber 303 was about 34 m³. The dehumidifier 307, the safety cabinet 201, and a dehumidifier terminal box 309 were disposed in the clean room 301. The safety cabinet 201 was an indoor circulation type class II anti-biohazard cabinet.

The mixed gas supply unit 1, the humidifying unit 103, the information acquisition unit 109, and the circulation fan 225 were disposed in the clean room 301. The mixed gas supply unit 1 was located at the center of the clean room 301.

The first solution 23 was an aqueous solution containing 40 w/v % of acetic acid. The amount of the first solution 23 was 150 ml. The flow rate of the air 18 was 10 L/min. The second solution was an aqueous solution containing 2 w/v % of hydrogen peroxide.

The BI were placed at the locations of P1 to P7 shown in FIG. 10. P1 was situated at a blow-port of the work space 205 in the safety cabinet 201, which was downstream of the HEPA 213. P2 was situated on an exhaust side of the safety cabinet 201. P3 was situated at a location between the safety cabinet 201 and a wall at the back of the safety cabinet 201. P4 was situated inside the dehumidifier terminal box 309. It was difficult to circulate air between inside and outside of the dehumidifier terminal box 309. P5 was situated on a surface of a wall of the clean room 301. P6 was situated on a surface of a wall of the front chamber 303. P7 was situated inside the clean room 301.

Firstly, humidity of the clean room 301 and the front chamber 303 was decreased to 50 to 60% RH by using the dehumidifier 307. Then, the space decontamination method was performed by supplying the mixed gas 25 by using the mixed gas supply unit 1 and by humidifying the space by using the humidifying unit 103. The safety cabinet 201 was operated when performing the space decontamination method. Decontamination gas circulated inside the safety cabinet 201 by having the safety cabinet 201 operated. The decontamination gas contains acetic acid and hydrogen peroxide.

When performing the space decontamination method, the humidity in the interior of the clean room 301 and the front chamber 303 was controlled to be about 75% RH by controlling the humidifying unit 103. When performing the space decontamination method, the dehumidifier 307 was operated intermittently. The dehumidifier 307 may be operated continuously. If an attempt was made to control the humidity at about 75% RH when the dehumidifier 307 was operated intermittently or continuously, the amount of spray by the humidifying unit 103 increases compared with a case where the dehumidifier 307 is not operated. Although the life of hydrogen peroxide in the atmosphere is short, the concentration of hydrogen peroxide in the clean room 301 and the front chamber 303 can be increased by increasing the amount of spray by the humidifying unit 103.

When performing the space decontamination method, air in the interior of the clean room 301 was agitated by the circulation fan 225. The humidifying unit 103 and the mixed gas supply unit 1 were situated in the path of wind sent out by the circulation fan 225.

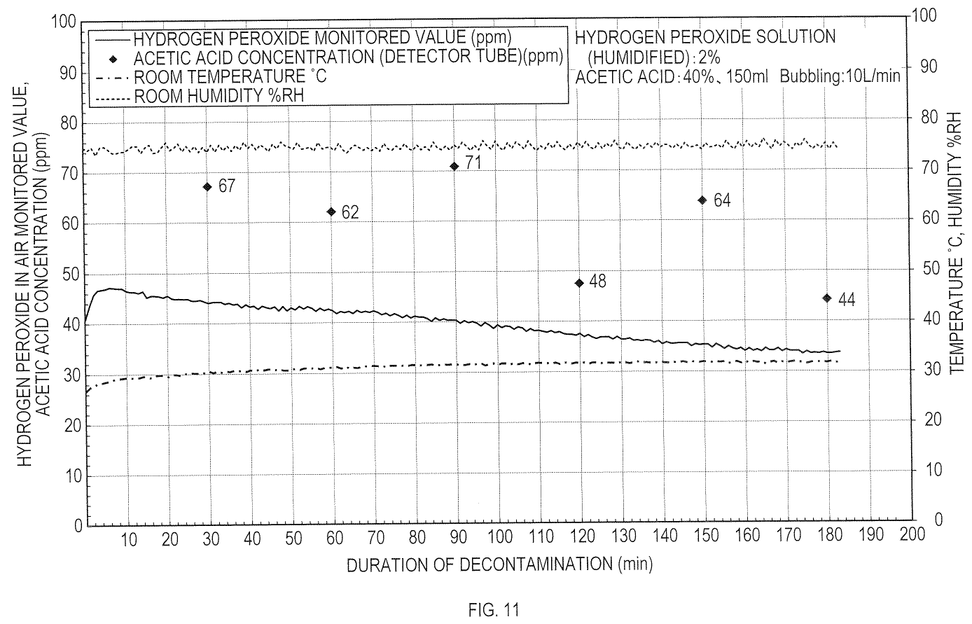

FIG. 11 shows transitions of concentration of hydrogen peroxide, concentration of acetic acid, temperature, and humidity in the interior of the clean room 301 when performing the space decontamination method. The horizontal axis of FIG. 11 represents the decontamination time. The decontamination time is a time elapsed since the time at which the humidity in the interior of the clean room 301 reached 75% RH. In a section where the decontamination time was 30 to 90 minutes, the average concentration of hydrogen peroxide was 42 ppm and the average concentration of acetic acid was 67 ppm in the interior of the clean room 301.

The BI were respectively recovered from the locations of P1, P2, P3, and P4 at 180 minutes of the decontamination time. The BI were also recovered from the location of P7 respectively at 60 minutes, 90 minutes, 120 minutes, 150 minutes, and 180 minutes of the decontamination time. Then, the recovered BI were cultivated for seven days in the dedicated culture media under a proper temperature to confirm whether the BI were negative or positive. The results are shown in Table 5. Three BI were recovered for one condition.

TABLE 5

| | Decontamination Time (min) | | | | |
|---|---|---|---|---|---|
| BI | 60 | 90 | 120 | 150 | 180 |
| P1 | | | | | − |
| | | | | | − |
| | | | | | − |
| P2 | | | | | − |
| | | | | | − |
| | | | | | − |
| P3 | | | | | − |
| | | | | | − |
| | | | | | − |
| P4 | | | | | − |
| | | | | | − |
| | | | | | − |
| P7 | + | − | − | − | − |
| | + | − | − | − | − |
| | + | − | − | − | − |

(+positive −negative)

As shown in Table 5, the BI were negative at all of the locations of P1 to P4. At the location of P7, the BI were negative when the decontamination time was 90 to 180 minutes. The decontamination gas inside the clean room 301 enters from a lower part of a work surface in the front surface of the safety cabinet 20 and reaches an exhaust port and the HEPA blow-off port at the work surface separately. The result that the BI were negative at the locations of P1 and P2 means that decontamination was also successful in the interior of the safety cabinet 201 and a filter where the decontamination gas passes through. Accordingly, it could be confirmed that the decontamination gas was highly permeable and can even permeate into the filter.

The BI disposed at the location of P4, where it was difficult to circulate air, was also confirmed negative. This means that the decontamination gas was highly permeable.

### (4-6) Comparative Example 3

The space decontamination method was performed basically in the same manner as in Example 3. However, in Comparative Example 3, the mixed gas 25 was not supplied to the interior of the clean room 301. In other words, in Comparative Example 3, acetic acid was not supplied to the interior of the clean room 301.

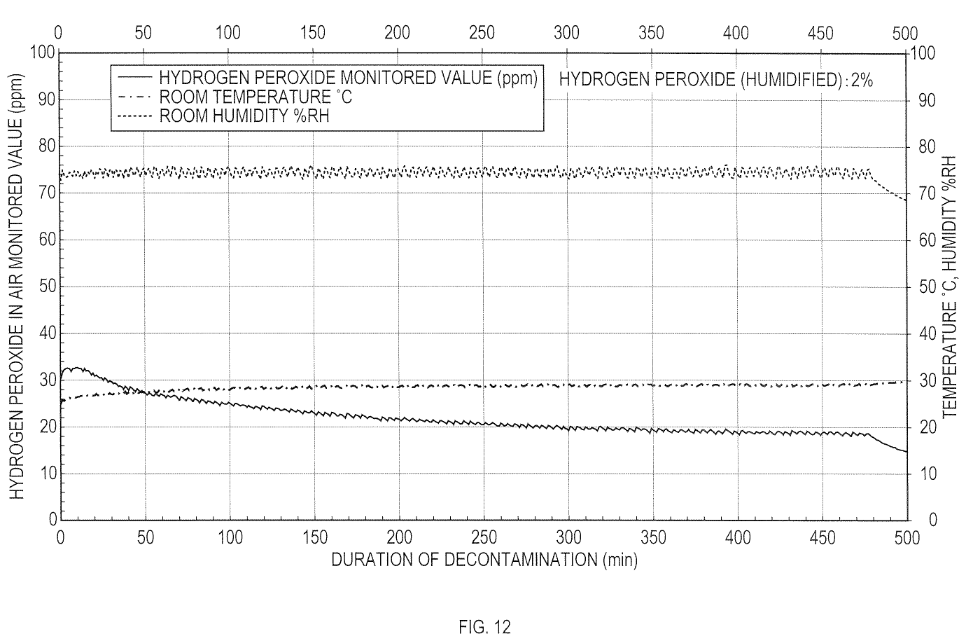

FIG. 12 shows transitions of the concentration of hydrogen peroxide, the concentration of acetic acid, temperature, and humidity in the interior of the clean room 301 when performing the space decontamination method. The horizontal axis of FIG. 12 represents the decontamination time. The decontamination time is a time elapsed since the time at which the humidity in the interior of the clean room 301 reached 75% RH. In a section where the decontamination time was 0 to 480 minutes, the average concentration of hydrogen peroxide was 22 ppm in the interior of the clean room 301.

The BI were respectively recovered from the locations of P1, P2, P3, P4, P5, and P6 at 480 minutes of the decontamination time. Then, the recovered BI were cultivated for seven days in the dedicated culture media under a proper temperature to confirm whether the BI were negative or positive. The results were shown in Table 6. One BI was recovered for one condition.

TABLE 6

| | Decontamination Time (min) |
|---|---|
| BI | 480 |
| P1 | + |
| P2 | + |
| P3 | − |
| P4 | + |
| P5 | − |
| P6 | − |

(+positive −negative)

As shown in Table 6, the BI were positive at the locations of P1, P2, and P4.

### (4-7) Example 4

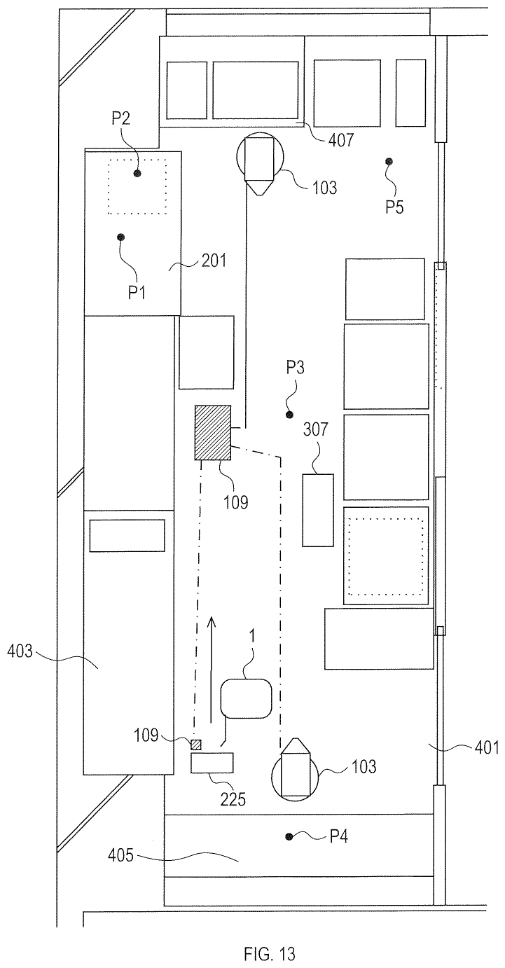

The space decontamination method was performed in a microorganism examination room 401 shown in FIG. 13. All entrances, exits, and windows of the microorganism examination room 401 were closed. Laboratory tables 403, 405, a sink 407, the safety cabinet 201, and the dehumidifier 307 were disposed in the microorganism examination room 401.

The mixed gas supply unit 1, two humidifying units 103, the information acquisition unit 109, and the circulation fan 225 were disposed in the microorganism examination room 401. The humidifying units 103 were the sprayers. The first solution 23 was an aqueous solution containing 40 w/v % of acetic acid. The amount of the first solution 23 was 150 ml. The flow rate of the air 18 was 15 L/min. The second solution was an aqueous solution containing 2 w/v % of hydrogen peroxide.

The BI were placed at the locations of P1 to P5 shown in FIG. 13. The BI were the same as those used in Example 1. P1 was situated at the location of the blow-off port of the work space 205 in the safety cabinet 201, which was downstream of the HEPA 213. P2 was situated on an exhaust side of the safety cabinet 201.

P3 and P5 were situated on the floor. P4 was situated at the back of the interior of a thermostatic bath which is not illustrated. The thermostatic bath was placed on the laboratory table 405. The door of the thermostatic bath was about 2 cm open. It was difficult to circulate air between inside and outside of the thermostatic bath.

The space decontamination method was performed by supplying the mixed gas 25 by using the mixed gas supply unit 1 and by humidifying the space by using the humidifying unit 103. The safety cabinet 201 was operated when performing the space decontamination method. Decontamination gas circulated inside the safety cabinet 201 by having the safety cabinet 201 operated.

When performing the space decontamination method, the humidity in the interior of the microorganism examination room 401 was controlled to be about 75% RH by controlling the humidifying unit 103. When performing the space decontamination method, air in the interior of the microorganism examination room 401 was agitated by the circulation fan 225. The mixed gas supply unit 1 was situated in the path of wind sent out by the circulation fan 225.

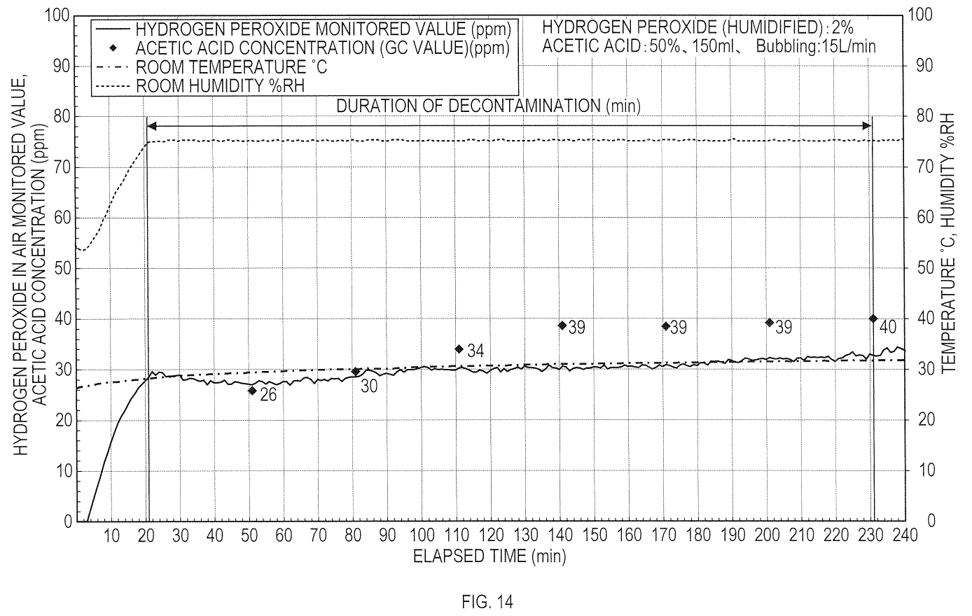

FIG. 14 shows transitions of concentration of hydrogen peroxide, concentration of acetic acid, temperature, and humidity in the interior of the microorganism examination room 401 when performing the space decontamination method. The horizontal axis of FIG. 14 represents time elapsed since the time at which humidification and supply of the mixed gas 25 were initiated. In a section where the decontamination time was 30 to 120 minutes, the average concentration of hydrogen peroxide was 30 ppm and the average concentration of acetic acid was 35 ppm in the interior of the microorganism examination room 401. The decontamination time is a time elapsed since the time at which the humidity of the interior of the microorganism examination room 401 reached 75% RH.

The BI were respectively recovered from the locations of P1, P2, P3, and P4 at 210 minutes of the decontamination time. The BI were also recovered from the location of P5 respectively at 90 minutes, 120 minutes, 150 minutes, 180 minutes, and 210 minutes of the decontamination time. Then, the recovered BI were cultivated for seven days in the dedicated culture media under a proper temperature to confirm whether the BI were negative or positive. The results are shown in Table 7. Three BI were recovered for one condition.

TABLE 7

| | Decontamination Time (min) | | | | |
|---|---|---|---|---|---|
| BI | 90 | 120 | 150 | 180 | 210 |
| P1 | | | | | − |
| | | | | | − |
| | | | | | − |
| P2 | | | | | − |
| | | | | | − |
| | | | | | − |
| P3 | | | | | − |
| | | | | | − |
| | | | | | − |
| P4 | | | | | − |
| | | | | | − |
| | | | | | − |
| P5 | + | + | − | − | − |
| | + | − | − | − | − |
| | − | − | − | − | − |

(+positive −negative)

As shown in Table 5, more than half of the BI were negative and decontamination was successful at all locations when the decontamination time was 120 minutes or more. The decontamination gas inside the clean room 301 enters from a lower part of the work surface in the front surface of a safety cabinet 20 and separately reaches the exhaust port and the HEPA blow-off port at the work surface. The result that the BI were negative at the locations of P1 and P2 means that decontamination was also successful in the interior of the safety cabinet 201 and the filter where the decontamination gas passes through. Accordingly, it was confirmed that the decontamination gas was highly permeable and can even permeate into the filter.

The BI disposed at the location of P4, where it was difficult to circulate air, was also negative. This means that the decontamination gas was highly permeable.

### 5. Other Embodiments

The embodiments of the present disclosure have been described above. The present disclosure is nevertheless not limited to the above-described embodiments and can be practiced in various modes.

(1) In the above Example 2, the space inside the safety cabinet was decontaminated. The present disclosure is nevertheless not limited thereto. For example, spaces inside an isolator, a restricted-access barrier system, or the like may be decontaminated.

(2) The control unit 107 and its procedures described in the present disclosure may be implemented by a dedicated computer provided with a processor programed to perform one or more functions embodied by a computer program and a memory. Alternatively, the control unit 107 and its procedures described in the present disclosure may be implemented by a dedicated computer provided with a processor including one or more dedicated hardware logic circuits. Or, the control unit 107 and its procedures described in the present disclosure may be implemented by one or more dedicated computers provided with a processor programed to perform one or more functions and a memory in combination with a processor provided with one or more hardware logic circuits. The computer program may be stored in a computer readable non-transitory tangible storage medium as instructions executed by the computer. Softwares may not necessarily be included in the procedures of implementing the functions of each parts included in the control unit 107. All of the functions may be implemented by one or more hardwares.

(3) Functions of one element in each of the aforementioned embodiments may be achieved by two or more elements; or functions of two or more elements may be performed by one element. A part of the configuration of each of the aforementioned embodiments may be omitted. At least a part of the configuration of each of the aforementioned embodiments may be added to or replaced with another part of the configuration of the aforementioned embodiments.

(4) In addition to the space decontamination device 101 mentioned above, the present disclosure may be implemented in various forms such as a system including the space decontamination device 101 as an element, a program to cause a computer to function as the control unit 107, a non-transitory tangible storage medium such as a semiconductor memory storing this program, and a method of manufacturing a space decontamination device.

### [Technical Ideas Disclosed in the Specification]

### [Item 1]

Item 1 is a space decontamination method. The method includes supplying a mixed gas containing air and acetic acid to a space, and humidifying the space by using an aqueous solution containing hydrogen peroxide.

### [Item 2]

Item 2 is the space decontamination method described in Item 1, in which the mixed gas is generated by sending air into the solution containing acetic acid and performing bubbling.

### [Item 3]

Item 3 is the space decontamination method described in Item 1 or Item 2, in which humidity of the space is 60% RH or higher and 80% RH or lower when performing the space decontamination method.

### [Item 4]

Item 4 is the space decontamination method described in any one of Items 1 to 3, in which concentration of acetic acid in the space is 10 ppm or higher and 100 ppm or lower and concentration of hydrogen peroxide in the space is 10 ppm or higher and 100 ppm or lower when performing the space decontamination method.

### [Item 5]

Item 5 is a space decontamination device including a mixed gas supply unit configured to supply a space with a mixed gas containing air and acetic acid; and a humidifying unit configured to humidify the space by using an aqueous solution containing hydrogen peroxide.

### [Item 6]

Item 6 is the space decontamination device described in Item 5, in which the mixed gas supply unit is configured to generate the mixed gas by sending air into a solution containing acetic acid and performing bubbling.

1. A space decontamination method comprising:
generating a mixed gas containing air and acetic acid by using a solution containing acetic acid (excluding a case where the solution contains peracetic acid and hydrogen peroxide);
supplying the mixed gas to a space; and
humidifying the space by using an aqueous solution containing hydrogen peroxide.

2. The space decontamination method according to claim 1,
wherein the mixed gas is generated by sending air into the solution containing acetic acid and performing bubbling.

3. The space decontamination method according to claim 1,
wherein humidity of the space is 60% RH or higher and 80% RH or lower when performing the space decontamination method.

4. The space decontamination method according to claim 1,
wherein concentration of acetic acid in the space is 10 ppm or higher and 100 ppm or lower and concentration of hydrogen peroxide in the space is 10 ppm or higher and 100 ppm or lower when performing the space decontamination method.

5. A space decontamination device comprising:
a mixed gas supply unit configured to supply a mixed gas containing air and acetic acid to a space; and
a humidifying unit configured to humidify the space by using an aqueous solution containing hydrogen peroxide,
wherein the mixed gas supply unit is configured to generate the mixed gas by using a solution containing acetic acid (excluding a case where the solution contains peracetic acid and hydrogen peroxide.

6. The space decontamination device according to claim 5,
wherein the mixed gas supply unit is configured to generate the mixed gas by sending air into the solution containing acetic acid and performing bubbling.
